(19) United States
(12) Patent Application Publication
(10) Pub. No.: US 2026/0159491 A1
(43) Pub. Date: Jun. 11, 2026

(54) METHOD FOR PRODUCING AN AQUEOUS 5-HYDROXYMETHYLFURFURAL (5-HMF) SOLUTION

(71) Applicant: IFP Energies nouvelles, Rueil-Malmaison Cedex (FR)

(72) Inventors: Marc JACQUIN, Rueil-Malmaison Cedex (FR); Maria FERNANDEZ ESPADA PASTOR, Rueil-Malmaison Cedex (FR); Kim LARMIER, Rueil-Malmaison Cedex (FR); Hélène LORCET, Rueil-Malmaison Cedex (FR); Thi Bich Ngoc DANG, Rueil-Malmaison Cedex (FR)

(73) Assignee: IFP Energies nouvelles, Rueil-Malmaison Cedex (FR)

(21) Appl. No.: 18/723,261

(22) PCT Filed: Dec. 6, 2022
(86) PCT No.: PCT/EP2022/084586

§ 371 Date: Jun. 21, 2024

(30) Foreign Application Priority Data
Dec. 23, 2021 (FR) FR2114335

(51) Int. Cl. C07D 307/50; B01D 3/00; B01D 11/04

(52) U.S. Cl. CPC C07D 307/50; B01D 3/009; B01D 11/04

(57) ABSTRACT

The invention relates to a process for producing an aqueous solution of 5-hydroxymethylfurfural (5-HMF), successively involving a) placing a feedstock containing 5-HMF and dimethyl sulfoxide (DMSO) in contact with an intermediate aqueous back-extract from a backwashing step c), b) a liquid-liquid extraction with an organic solvent followed by a step c) of backwashing with an aqueous solvent to obtain an organic raffinate rich in 5-HMF and solvent. The raffinate is then subjected to a concentration step d) and then a hydrodistillation step e) to obtain an aqueous solution of 5-HMF.

### TECHNICAL FIELD

The invention relates to a process for producing an aqueous solution of 5-hydroxymethylfurfural (5-HMF).

### PRIOR ART

5-HMF is an advantageous compound resulting from biomass, which can be exploited in many fields, notably in pharmaceuticals, agrochemistry or speciality chemistry. The production of 5-HMF by dehydration of sugars has been known for many years and has formed the subject of a large number of research studies. There are numerous dehydration conditions, and mention may notably be made, by way of example, of the following methods:
5-HMF can be obtained in aqueous medium, generally in the presence of an acid catalyst. This acid catalyst makes it possible to dehydrate the C6 sugar (in particular fructose) to give 5-HMF, but also catalyzes the rehydration of 5-HMF to give formic acid and levulinic acid, which is very harmful to the yield.
5-HMF can also be obtained in nonaqueous polar protic medium, with solvents such as methanol, ethanol or acetic acid, and in the presence of an acid catalyst. Under these conditions, 5-HMF is obtained as a mixture with an ether or ester derivative of 5-HMF, depending on the reaction medium used. The formation of these byproducts is due to the reaction of 5-HMF with the reaction solvent in acidic medium.
Patent application WO 2007/104514 describes the synthesis of 5-HMF by dehydration of sugar using methanol or ethanol as solvent in the presence of an acid catalyst. In this case, the presence of said catalyst also catalyzes the etherification reaction of 5-HMF with the alcohol to give a mixture of 5-HMF and of its methyl or ethyl ether form, depending on the alcohol used as solvent.
5-HMF can also be produced in polar aprotic medium, with or without an acid catalyst. Mention may more particularly be made of the use of dimethyl sulfoxide (DMSO) which, with or without an acid catalyst, makes it possible to produce 5-HMF in very good yields, and without the undesirable reactions listed above.

Furthermore, whatever the synthesis medium (water, methanol, DMSO, and the like), polymeric byproducts called humins are formed during the production of 5-HMF (van Dam, H. E. ; Kieboom, A. P. G. ; van Bekkum, H. (1986), The Conversion of Fructose and Glucose in Acidic Media: Formation of Hydroxymethylfurfural, In: Starch-Stärke, vol. 38, No. 3, pages 95-101).

The synthesis of 5-HMF in a medium such as DMSO is particularly advantageous, as it makes it possible to obtain 5-HMF in its alcohol form (rather than the ether form) in very good yields. Nevertheless, the physicochemical properties of DMSO (or any other polar aprotic solvent) make it very difficult to separate from 5-HMF by the usual methods known to those skilled in the art.

One known method for isolating 5-HMF from DMSO is liquid-liquid extraction, followed by crystallization of the extract, as described in patent FR 2 669 635. The Applicant has already proposed an improvement to the process described in patent FR 2669635, which was the subject of patent FR 1758605. This improvement is based on modifying the extraction step, notably by adding a water backwashing step, and recycling the backwashing water into the optional filtration step. This improvement allows the purity of 5-HMF to be increased without loss of yield of the product of interest, and allows the 5-HMF crystallization step to be performed under more favorable conditions.

Nevertheless, despite the improvements afforded by patent FR 1758605, 5-HMF crystallization remains a costly operation. The high production cost of 5-HMF limits its use, and the development of a cost-saving process is necessary.

The Applicant has discovered a process which allows 5-HMF to be recovered not in crystallized form but in aqueous solution, opening up new possibilities for the exploitation of 5-HMF in various applications, or for further transformations which could not be performed either in DMSO or in the extraction solvent. Moreover, the process according to the invention thus allows 5-HMF to be recovered in aqueous solution, while at the same time limiting the operating costs, water discharges and hence the environmental impact of said process.

### SUMMARY OF THE INVENTION

One subject of the present invention relates to a process for producing an aqueous solution of 5-HMF.

More particularly, the invention relates to a process for producing an aqueous solution of 5-hydroxymethylfurfural (5-HMF), said process comprising the following steps:
a step a) of placing a feedstock comprising 5-HMF and dimethyl sulfoxide (DMSO) in contact with at least a fraction of an intermediate aqueous back-extract, advantageously obtained from step c), so as to obtain at least one aqueous mixture,
a step b) of liquid-liquid extraction of the aqueous mixture obtained on conclusion of step a) in the presence of an extraction solvent, so as to produce an aqueous raffinate and an intermediate organic extract, and then
a step c) of backwashing with an aqueous solvent, so as to produce the intermediate aqueous back-extract and an organic raffinate which comprises 5-HMF and an organic solvent,
a step d) of concentrating the organic raffinate obtained from step c) by removing at least part of the organic solvent, producing a concentrated organic extract, comprising 5-HMF, preferably in a content greater than or equal to 40% by weight, and residual organic solvent, preferably in a content less than or equal to 60% by weight, and a stream comprising organic solvent,
a hydrodistillation step e) performed by distilling the concentrated organic extract obtained from step d) in the presence of water, to produce an aqueous solution of 5-HMF and a stream comprising organic solvent,
an optional step f) of treating the water-DMSO mixtures produced within the process, allowing the production of an aqueous effluent, which may be used totally or partly in the backwashing step c), and/or in step e).

### DESCRIPTION OF THE EMBODIMENTS

It is specified that, throughout this description, the expression “between . . . and . . . ” should be understood as including the limits mentioned.

For the purposes of the present invention, the various embodiments presented may be used alone or in combination with each other, without any limit to the combinations.

For the purposes of the present invention, the various ranges of parameters for a given step, such as the pressure ranges and the temperature ranges, can be used alone or in combination.

For example, for the purposes of the present invention, a preferred range of pressure values can be combined with a more preferred range of temperature values.

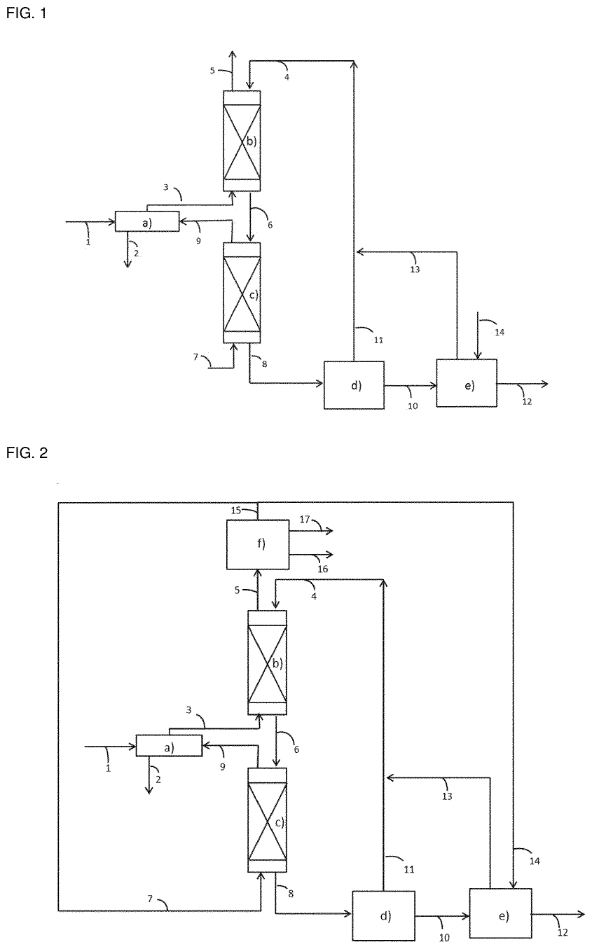

For better understanding of the invention, numerical references appearing in the figures are mentioned below to denote various elements of the process, without this constituting a limitation to the particular embodiments illustrated in FIGS. 1 and 2.

### Optional Step of Sugar Dehydration to 5-HMF

Advantageously, the feedstock 1 comprising 5-HMF and dimethyl sulfoxide (DMSO) introduced in step a) according to the invention can be obtained during a step of sugar dehydration to 5-HMF, very advantageously located upstream of step a) according to the invention, by placing a sugar feedstock comprising one or more sugars in contact with DMSO and an acidic dehydration catalyst so as to produce an effluent comprising at least 5-HMF and DMSO and advantageously corresponding to the feedstock 1 of the process according to the invention introduced in the mixing step a). The process according to the invention may thus optionally comprise a step of sugar dehydration to 5-HMF, located upstream of step a).

The term “acidic dehydration catalyst” means any Brønsted acid catalyst chosen from organic or inorganic, homogeneous or heterogeneous Brønsted acids which can induce the dehydration of sugars to 5-HMF.

Preferably, the acidic dehydration catalyst is a Brønsted acid with a pKa in DMSO of between 0 and 5.0, preferably between 0.5 and 4.0 and more preferably between 1.0 and 3.0. Said pKa values are as defined in the article by F. G. Bordwell et al. (J. Am. Chem. Soc., 1991, 113, 8398-8401).

Preferably, the acidic dehydration catalyst is chosen from HF, HCl, HBr, HI, H₂SO₃, H₂SO₄, H₃PO₂, H₃PO₄, HNO₂, HNO₃, H₂WO₄, H₄SiW₁₂O₄₀, H₃PW₁₂O₄₀, (NH₄)₆(W₁₂O₄₀)·XH₂O, H₄SiMO₁₂O₄₀, H₃PMo₁₂O₄₀, (NH₄)₆MO₇O₂₄·xH₂O, H₂MOO₄, HReO₄, H₂CrO₄, H₂SnO₃, H₄SiO₄, H₃BO₃, HClO₄, HBF₄, HSbF₅, HPF₆, H₂FO₃P, ClSO₃H, FSO₃H, HN(SO₂F)₂, HIO₃, BF₃, AlCl₃, Al(OTf)₃, FeCl₃, ZnCl₂, SnCl₂, CrCl₃, CeCl₃, ErCl₃, formic acid, acetic acid, trifluoroacetic acid, lactic acid, levulinic acid, methanesulfinic acid, methanesulfonic acid, trifluoromethanesulfonic acid, bis(trifluoromethanesulfonyl)amine, benzoic acid, para-toluenesulfonic acid, 4-biphenylsulfonic acid, diphenyl phosphate and 1,1′-binaphthyl-2,2′-diyl hydrogen phosphate. Preferably, the acidic dehydration catalyst is chosen from HCl, H₂SO₄, H₃PO₂, H₃PO₄, HNO₃, AlCl₃, acetic acid, trifluoroacetic acid, methanesulfinic acid, methanesulfonic acid and trifluoromethanesulfonic acid.

The term “sugar” denotes a sugar containing 6 carbon atoms (hexoses), but this does not exclude the presence in the feedstock of sugars containing 5 carbon atoms (pentoses), in the form of oligosaccharides and monosaccharides. In particular, the term “sugar” denotes glucose or fructose, alone or as a mixture, sucrose, and also oligosaccharides such as cellobiose, maltose, cellulose or even inulin.

The sugar feedstock used may be sugar in solid form, or an aqueous sugar solution. By way of example, sucrose is generally produced in the form of a solid, while glucose or fructose, alone or as a mixture, are generally produced in the form of an aqueous solution (syrup), for example at 70% by weight of sugar.

The optional dehydration step is performed at a temperature of between 50 and 150° C., preferably between 60 and 140° C., preferably between 70 and 130° C. and more preferably between 80 and 120° C. Preferably, the optional dehydration step is performed at a pressure of between 1 and 0.001 MPa, preferably between 0.1 and 0.01 MPa. Depending on the pressure and temperature conditions, the reaction medium is above or below the bubble point of the mixture. The term “bubble point” denotes the pressure and temperature conditions under which the first gas bubbles are seen in a liquid. When the reaction medium is above the bubble point of the mixture, the vapor phase can be withdrawn from the reactor, optionally rectified, and condensed to form condensates which can be sent to an optional step f) for processing the water-DMSO mixtures.

Preferably, the acidic dehydration catalyst is introduced into the dehydration step in a mole ratio of the catalyst relative to the sugar feedstock, denoted Acid/Sugar, expressed as a molar percentage (mol %), of between 0.01 and 10 mol %, preferably between 0.05 and 8 mol %, preferably between 0.1 and 6 mol %, preferably between 0.2 and 5 mol %, more preferably between 0.3 and 4 mol % and very preferably between 0.5 and 3 mol %.

Advantageously, the effluent obtained on conclusion of the optional dehydration step comprises 5-HMF and DMSO. DMSO generally represents between 30% and 95% by weight of the effluent resulting from the dehydration step and treated in step a) of the process according to the invention, preferably between 40% and 90% by weight, preferably between 50% and 90% by weight, more preferably between 55% and 85% by weight.

5-HMF represents more than 1% by weight of the effluent resulting from the optional dehydration step and treated in step a) of the process according to the invention, preferably more than 10% by weight, preferably more than 15% by weight and preferably less than 50% by weight, preferably less than 40% by weight, more preferably less than 30% by weight.

Furthermore, said effluent from the optional dehydration step may contain water even before being mixed in step a) with the intermediate aqueous back-extract 9. Said water may be derived from the dehydration step: for example, water is formed during the dehydration reaction of sugar to 5-HMF (3 mol of water generated per mole of 5-HMF produced). This water may also have been introduced with the sugar, in the case where, for practical reasons, a sugar syrup, for example at about 70% by weight in water, is used. Advantageously, during the optional dehydration step, a water-DMSO mixture may be recovered in the vapor phase. Said water-DMSO mixture is advantageously sent to the optional step f). Thus, the effluent resulting from the optional dehydration step and introduced into step a) as feedstock 1 may contain water, in a proportion generally between 0.1% and 30% by weight, preferably between 0.1% and 15% by weight, more preferably between 0.1% and 10% by weight.

The effluent resulting from the optional dehydration step and introduced into step a) as feedstock 1 may also contain impurities, in particular humins. The term “humins” refers to all the undesirable polymeric compounds formed during the synthesis of 5-HMF. In particular, humins represent less than 30% by weight of the converted sugar feedstock, preferably less than 20% by weight.

The optional dehydration step may be performed according to various embodiments. Thus, the step may advantageously be performed batchwise or continuously. The addition of the sugar feedstock may be progressive (fed-batch) in the case of a batch process, or staged in different CSTR reactors (Continuously Stirred Tank Reactor) in series in the case of a continuous process. The process can be performed in a closed reaction chamber or in a semi-open reactor.

### Mixing Step a)

The process according to the invention comprises a step a) of placing in contact (or mixing) the feedstock 1, optionally from the dehydration step, with at least a fraction of an intermediate aqueous back-extract 9, in order to obtain at least one aqueous mixture 3. Advantageously, the intermediate aqueous back-extract 9 is obtained from step c) of the process according to the invention.

Preferably, 5-HMF represents more than 1% by weight of the feedstock 1 introduced into step a) of the process according to the invention, preferably more than 10% by weight, preferably more than 15% by weight and preferably less than 50% by weight, preferably less than 40% by weight, more preferably less than 30% by weight.

Preferably, DMSO represents between 30% and 95% by weight of the feedstock 1 introduced into step a), preferably between 40% and 90% by weight, preferably between 50% and 90% by weight, more preferably between 55% and 85% by weight.

The feedstock 1 introduced into step a) may also contain water, in a proportion preferably between 0.1% and 30% by weight, preferably between 0.1% and 15% by weight and more preferably between 0.1% and 10% by weight.

Feedstock 1 may also optionally contain humins. Humins represent, in particular, less than 30% by weight of feedstock 1, preferably less than 20% by weight.

The intermediate aqueous back-extract 9 or the fraction of intermediate aqueous back-extract 9 is advantageously obtained from step c). It comprises water, DMSO and optionally 5-HMF. Advantageously, said intermediate aqueous back-extract 9 contains more than 60% by weight of water, preferably more than 70% by weight of water and preferably more than 80% by weight of water.

Advantageously, the aqueous mixture 3 obtained on conclusion of step a) contains between 10% and 90% by weight of water, preferably between 20% and 80% by weight of water, more preferably between 40% and 75% by weight of water.

Preferably, step a) is performed at a temperature of from 0 to 60° C., preferably from 10 to 30° C. and generally at room temperature, i.e. between 18 and 25° C.

Step a) may optionally also be fed with an aqueous stream, for example with a fraction of the aqueous solvent used in the backwashing step c).

By increasing the water content during step a), for example by introducing at least a fraction of the intermediate aqueous back-extract 9, some of the humins that may be present in feedstock 1 can precipitate out. The mixture resulting from contact of said feedstock 1 with at least a fraction of the intermediate aqueous back-extract 9 can thus advantageously be subjected to a liquid-solid separation step, so as to obtain a liquid separated from suspended solid particles and a solid residue comprising humins and which is preferably eliminated from the process. Such an optional liquid-solid separation step thus allows precipitated humins to be removed. At least part of the liquid obtained is then advantageously sent to the liquid-liquid extraction step b), said part (or all) of the liquid advantageously sent to step b) corresponding to the aqueous mixture 3. When the amount of precipitated humins in the mixture is low (for example 1% by weight or less), the liquid-solid separation step is optional. This optional liquid-solid separation step is preferably performed at a temperature of between 0 and 60° C., preferably between 10 and 30° C., preferably between 15 and 25° C. and generally at room temperature (i.e. between 18 and 25° C.). The optional liquid-solid separation step is a simple solid-liquid separation and may be performed via any method known to those skilled in the art, for instance with a filter press, a belt filter, a clarifier, a decanter or a centrifuge, for example a plate centrifuge. Preferably, the liquid-solid separation step is a filtration, preferably performed with a filter press.

### Extraction Step B)

The process according to the invention comprises a step b) of liquid-liquid extraction of the aqueous mixture 3 obtained on conclusion of step a) in the presence of an extraction solvent 4, so as to produce an aqueous raffinate 5 and an intermediate organic extract 6.

The liquid-liquid extraction performed in step b) advantageously corresponds to washing the aqueous mixture with an organic extraction solvent. Preferably, the liquid-liquid extraction performed in step b) is a countercurrent extraction of the aqueous mixture 3 obtained in step a) with an extraction solvent. This technique is well known to those skilled in the art. The extraction can be performed, for example, in a mixer-decanter array, in a column filled with random or structured packing, in a plug-flow column, or else in a stirred column.

The liquid-liquid extraction step b) is advantageously performed at a temperature of between 0 and 60° C., preferably between 5 and 50° C., preferably between 10 and 40° C., more preferably between 15 and 30° C. and generally at room temperature (i.e. between 18 and 25° C.). The weight proportion (weight/weight) of extraction solvent relative to the aqueous mixture 3 is preferably from 0.2 to 5, preferably between 1 and 3, more preferably between 1.5 and 2.5.

The extraction solvent introduced into step b) is chosen from water-immiscible organic solvents, so as to form two liquid phases in the backwashing step c). This property is highly dependent on the relative proportion of the flow rates of feedstock, of back-extraction water and of extraction solvent used in the process.

In a nonlimiting manner, the extraction solvent is preferably chosen from chlorinated organic solvents, ethers, esters, ketones and aromatic compounds. Preferably, the extraction solvent is a chlorinated solvent containing between 1 and 10 carbon atoms, noted below as C1-C10, an ether containing between 2 and 10 carbon atoms (C2-C10), an ester containing between 4and 10 carbon atoms (C4-C10), a ketone containing between 3 and 10 carbon atoms (C3-C10), an aldehyde containing between 1 and 10 carbon atoms (C1-C10) or a C4-C10 aromatic compound. Preferably, the extraction solvent is chosen from dichloromethane, diethyl ether, diisopropyl ether, methyl ethyl ketone, methyl isopropyl ketone, methyl isobutyl ketone, thiophene, anisole and toluene. Very preferably, the extraction solvent is methyl isobutyl ketone.

Advantageously, the extraction solvent is chosen so as:
to have a very high difference in volatility with 5-HMF so as to facilitate its removal in step d) and to limit the degradation of 5-HMF, i.e. so as to have in step d) a vaporization rate which avoids degradation of 5-HMF and minimizes the amount of residual solvent to be removed in step e), while at the same time ensuring the absence of liquid phase separation when the concentrated organic extract is placed in contact with water in step e), and
to form in step e) a heterogeneous azeotrope with water, preferably solvent-rich, i.e. with more than 50% by weight of solvent, preferably with more than 60% by weight of solvent and more preferably with more than 70% by weight of solvent. Advantageously, said azeotrope of the water/extraction solvent mixture has a boiling temperature significantly lower than that of water, preferably at least 5° C. lower than the boiling temperature of water, preferably at least 8° C. lower than the boiling temperature of water and preferably at least 10° C. lower than the boiling temperature of water.

Advantageously, the organic solvent streams produced in the subsequent steps may be recycled into the extraction step b), as extraction solvent. These organic solvent streams may contain impurities which may have been generated during the implementation of the process. Advantageously, the organic solvent streams produced in the subsequent steps may be distilled, for example periodically, to avoid accumulation of said impurities.

Step b) thus makes it possible to obtain, on the one hand, an aqueous stream depleted in 5-HMF, called aqueous raffinate 5, which contains a large proportion of the DMSO initially contained in the feedstock, and on the other hand an organic stream enriched in 5-HMF, called intermediate organic extract 6, which contains a large proportion of the 5-HMF, initially contained in feedstock 1, and the extraction solvent. This intermediate organic extract 6 may also contain DMSO. Preferably, said intermediate organic extract preferably contains 5-HMF and DMSO in a 5-HMF/DMSO weight ratio of between 50/50 and 95/05, preferably between 55/45 and 90/10, preferably between 60/40 and 85/15 and more preferably between 65/35 and 80/20.

Advantageously, the intermediate organic extract 6 is sent directly to the backwashing step c).

### Backwashing Step C)

The process according to the invention comprises a step c) of backwashing, advantageously of the intermediate organic extract 6, with an aqueous solvent 7, so as to produce an intermediate aqueous back-extract 9 and an organic raffinate 8 comprising 5-HMF and an organic solvent. The intermediate aqueous back-extract 9 is advantageously sent partly or totally into step a). The organic solvent is in particular at least partly composed of extraction solvent and may optionally comprise DMSO, preferably in small amounts.

The introduction of an aqueous solvent in step c) is performed so as to perform backwashing, according to the general knowledge of those skilled in the art. The introduction of the aqueous solvent is performed such that the amount of aqueous solvent is as low as possible in order to reduce costs, but sufficient to ensure a low DMSO weight content in the organic raffinate 8, preferably less than or equal to 20.0% by weight relative to the weight of 5-HMF, preferentially less than or equal to 15.0% by weight relative to the weight of 5-HMF, preferably between 0.01% and 15.0% by weight relative to the weight of 5-HMF, very preferably between 0.01% and 10.0% by weight relative to the weight of 5-HMF.

Advantageously, the aqueous backwashing solvent introduced in step c) comprises more than 95% by weight of water, preferably more than 98% by weight of water (100% being the maximum). The aqueous solvent may optionally comprise DMSO. The backwashing efficacy is all the higher the lower the amount of DMSO present in the aqueous backwashing solvent. Preferably, the aqueous solvent may comprise DMSO, preferably less than 1.0% by weight of DMSO, more preferably less than 0.1% by weight of DMSO. Advantageously, the aqueous backwashing solvent is derived from an optional step f) of treating water-DMSO mixtures produced within the process. In a preferred embodiment of the invention, the aqueous raffinate 5 composed of water and DMSO, produced in step b), is treated, advantageously in an optional step f) which in particular comprises distillation. The water-rich distillate thus obtained from this optional step f) is advantageously used as an aqueous backwashing solvent in step c); said water-rich distillate may also contain a residual amount of DMSO, preferably less than 1% by weight and preferably less than 0.1% by weight of DMSO. The residual amount of DMSO in the distillate is proportionately lower the more efficiently the distillation of optional step f) is performed, in particular with a number of distillation stages greater than 10 and suitable reboiling and reflux rates.

The backwashing step c) is advantageously a liquid-liquid extraction of an organic stream, in particular the intermediate organic extract 6 obtained in step b) in countercurrent to the aqueous solvent 7. This technique is well known to those skilled in the art. The extraction can be performed, for example, in a mixer-decanter array, in a column filled with random or structured packing, in a plug-flow column, or else in a stirred column.

Step c) is preferably performed at a temperature of between 0 and 60° C., preferably between 5 and 50° C., preferably between 10 and 40° C., more preferably between 15 and 30° C. and generally at room temperature (i.e. between 18 and 25° C.).

The weight ratio (weight/weight) of aqueous solvent relative to the intermediate organic extract 6 is preferably from 0.04 to 5, preferably between 0.07 and 3, more preferably between 0.1 and 1.

Step c) produces an aqueous stream advantageously enriched in DMSO, referred to as intermediate aqueous back-extract 9, preferably containing at least 60% by weight of water, preferably at least 80% by weight of water, and an organic raffinate 8, advantageously depleted in DMSO. Said intermediate aqueous back-extract 9 is advantageously sent, partly or preferably totally, to step a). The organic raffinate 8 obtained has a DMSO weight content preferably less than or equal to 20.0% by weight relative to the weight of 5-HMF, preferably less than or equal to 15.0% by weight relative to the weight of 5-HMF, preferably less than or equal to 5.0% by weight relative to the weight of 5-HMF, preferably less than or equal to 4.0% by weight relative to the weight of 5-HMF, preferably less than or equal to 3.0% by weight relative to the weight of 5-HMF.

According to the invention, the organic raffinate 8 produced in step c) is sent to the concentration step d).

### Concentration Step d)

The process according to the invention comprises a step d) of concentrating the organic raffinate 8 obtained from step c), by removal of part of the organic solvent, producing a concentrated organic extract 10, comprising 5-HMF and residual organic solvent, and a stream 11 comprising, preferably consisting of, organic solvent, said organic solvent advantageously being composed totally or partly of extraction solvent and optionally DMSO.

Preferably, stream 11 comprising organic solvent is recycled, totally or partly, into extraction step b).

Preferably, in step d), removal of part of the organic solvent is performed by vaporization, for example in a distillation column at atmospheric pressure or under vacuum, in an evaporator, or via any method known to those skilled in the art.

According to this preferred embodiment, vaporization of the organic solvent is advantageously performed at atmospheric pressure or under vacuum, preferably at a pressure of between 0.1 and 0.01 MPa, preferentially under vacuum at a pressure of between 0.09 and 0.01 MPa, so as to limit the temperature of the liquid and thus the degradation of 5-HMF. Preferably, the temperature of the liquid is kept below 130° C., preferably kept below 100° C., more preferably kept below 70° C. The vacuum level to be applied to reach these temperatures is of course dependent on the organic solvent and more particularly on the extraction solvent used and the organic solvent vaporization ratio.

In a preferred embodiment, solvent vaporization is performed by multi-effect evaporation or mechanical vapor recompression, or any other method known to those skilled in the art, so as to reduce the operating costs associated with solvent evaporation while limiting the risk of degradation of the product of interest, i.e. 5-HMF. For example, in the case of a triple-effect evaporator, the liquid temperature is kept below 130° C. in the first effect, below 100° C. in the second effect, and below 70° C. in the third effect. Thus, the temperature of the liquid phase is progressively reduced as the 5-HMF is concentrated in the organic solvent, limiting any risk of degradation.

Step d) is performed with a vaporization mass ratio (or evaporation ratio), corresponding to the mass of organic solvent vaporized relative to the mass of organic raffinate 8 from step c) (more particularly the mass amount of stream 11 relative to the mass amount of organic raffinate 8), of at least 50%, preferably of at least 60%, preferably of at least 70%, preferably of at least 75%, preferably of at least 80%, preferably of at least 85%, preferably of at least 90%, and more preferably of at most 99%. Advantageously, the vaporization ratio is defined as a function of the extraction solvent so as not to degrade the 5-HMF, but also so as to minimize the amount of residual solvent to be removed in step e), while at the same time ensuring the absence of liquid phase separation (i.e. while ensuring that the liquid phase remains monophasic) when the concentrated organic extract 10 is placed in contact with water in step e).

According to the invention and in particular by means of the combination of all the operating conditions of step d) and the preceding steps a), b) and c), the concentrated organic extract 10 obtained on conclusion of step d) very advantageously has a 5-HMF content of at least 40% by weight relative to the weight of concentrated organic extract, preferably at least 50% by weight, preferably at least 60% by weight, and preferably not more than 95% by weight, preferably not more than 90% by weight and preferably not more than 85% by weight, relative to the weight of concentrated organic extract. In other words, the concentrated organic extract 10 preferably has a residual organic solvent content of at least 5% by weight relative to the weight of concentrated organic extract, preferably at least 10% by weight, and preferably not more than 60% by weight, preferably not more than 50% by weight, more preferably not more than 40% by weight, relative to the weight of concentrated organic extract 10.

Advantageously, the organic solvent vaporized during step d) forms a stream 11 comprising, preferably consisting of, organic solvent and is preferably recycled into extraction step b).

Advantageously, the concentrated organic extract 10 is sent to the hydrodistillation step e).

### Hydrodistillation Step E)

The process according to the invention comprises a hydrodistillation step e) performed by distilling the concentrated organic extract 10 from step d) in the presence of water, in order to produce an aqueous solution 12 of 5-HMF and a stream 13 comprising, preferably consisting of, organic solvent.

Hydrodistillation step e) advantageously enables the residual organic solvent not removed during step d) to be at least partly removed. The residual organic solvent removed during step e), i.e. stream 13, may advantageously be recycled into extraction step b) alone or as a mixture with stream 11 comprising organic solvent from step d).

Advantageously, an aqueous stream 14 feeds the hydrodistillation step e). The aqueous stream 14 introduced in step e) preferably contains more than 95% by weight of water, more preferably more than 98% by weight of water.

In a particular embodiment of the invention, the aqueous stream 14 is pure water, possibly external to the process, which allows the residual DMSO content in the aqueous 5-HMF solution 12 produced in step e) to be minimized even further.

In another particular embodiment of the invention, water isolated within the process is used to feed step e), allowing the process operating costs and its environmental impact to be limited. Typically, if the process includes the preparation of feedstock 1 and the sugar feedstock for the dehydration step is a sugar syrup at 70% by weight in water, about 1 ton of water is recovered on conclusion of the dehydration step (the feedstock water and the water produced during the dehydration reaction) per ton of 5-HMF produced. This water needs to be treated before being released into the environment. The process according to the invention can then advantageously use said water from the dehydration step to produce on conclusion of step e) an aqueous solution of 5-HMF concentrated preferably to 30% by weight or more, preferentially to 40% by weight or more, and thus reduce the reprocessing costs of the process and its environmental impact.

Advantageously, the aqueous stream 14 introduced into step e) may correspond to at least a fraction, optionally all, of the distillate produced in the optional step f). Said distillate may optionally contain a residual amount of DMSO.

Advantageously, during step e), the extraction solvent used in the process forms a heterogeneous azeotrope with water, said azeotrope preferably being rich in extraction solvent, preferably comprising more than 50% by weight of extraction solvent, preferably more than 60% by weight of extraction solvent and preferably more than 70% by weight of extraction solvent. Advantageously, said water/extraction solvent azeotrope has a boiling temperature significantly lower than that of water, preferably at least 5° C. lower than the boiling temperature of water, preferably at least 8° C. lower than the boiling temperature of water and preferably at least 10° C. lower than the boiling temperature of water.

Thus, after placing concentrated organic extract 10 in contact with aqueous stream 14, residual organic solvent contained in concentrated organic extract 10 may be readily removed without degradation of 5-HMF.

Hydrodistillation step e) may be performed at atmospheric pressure or under vacuum and in particular at a pressure of between 0.1 MPa and 0.001 MPa, preferably under vacuum at a pressure of between 0.08 and 0.005 MPa. Advantageously, the hydrodistillation step is performed under vacuum, in particular at a pressure of between 0.1 MPa and 0.001 MPa, preferably between 0.08 and 0.005 MPa, so as to facilitate removal of the residual organic solvent without degradation of the 5-HMF.

Advantageously, the hydrodistillation step e) is performed in a distillation column, preferably at a column bottom temperature of less than 140° C., preferably less than 130° C., preferably less than 120° C., preferably less than 110° C. and preferably less than 100° C., so as to facilitate removal of the residual organic solvent without degradation of the 5-HMF.

In one particular embodiment, concentrated organic extract 10 and aqueous stream 14 are mixed prior to introduction into a distillation column and the mixture is introduced at an intermediate point of the distillation column.

In another particular embodiment, the concentrated organic extract 10 is introduced into the upper part of the distillation column, preferably into the upper half of the distillation column, while the aqueous solvent is introduced into the lower part of the distillation column, preferably into the lower half of the distillation column. Mixing of the concentrated organic extract and the aqueous stream is then performed within the distillation column.

Given the formation of a heterogeneous azeotrope between water and the extraction solvent, condensation of the distillation column overhead vapors generates two liquid phases: a water-rich phase which may advantageously be returned to the column as reflux, and an organic solvent-rich phase which may advantageously be recycled into extraction step b).

According to the invention, the aqueous 5-HMF solution 12 obtained on conclusion of step e) has an amount of 5-HMF of at least 30% by weight, preferably at least 40% by weight, and preferably less than 90% by weight, preferably less than 85% by weight and preferably less than 80% by weight, the percentages being given by weight of 5-HMF relative to the weight of aqueous 5-HMF solution obtained on conclusion of step e).

The process according to the invention thus allows the production of an aqueous solution of 5-HMF very advantageously having a DMSO weight content of less than or equal to 10% by weight relative to the weight of 5-HMF, preferably less than or equal to 5% by weight relative to the weight of 5-HMF and preferably less than or equal to 3% by weight relative to the weight of 5-HMF.

### Optional Step F) of Treating the Water-DMSO Mixtures

The process according to the invention may comprise an optional step f) of treating the water-DMSO mixtures generated by the steps of the process according to the invention, to produce an aqueous effluent (also known as distillate), which may be totally or partly used in the backwashing step c) and/or in step e). This step may also produce a DMSO-rich stream 16 and an impurities stream 17.

The residual amount of DMSO in the aqueous effluent produced on conclusion of optional step f) is all the lower as distillation is performed in an efficient manner according to the knowledge of those skilled in the art.

The water-DMSO mixtures generated via the process denote in particular the aqueous raffinate 5 produced in step b) and optionally the water-DMSO mixture resulting from the optional step of dehydration of sugars to 5-HMF when the process incorporates such a step. Preferably, optional step f) of treating the water-DMSO mixtures involves a section for evaporating a water-DMSO mixture, to remove any impurities (stream 17), in particular heavy impurities such as humins, followed by a distillation section.

The evaporation section is operated at a temperature preferably between 80 and 120° C., preferentially between 100 and 110° C., and preferably at a pressure between 0.002 and 0.020 MPa, preferentially between 0.005 and 0.010 MPa. Preferably, the evaporation section uses a Thin Film Evaporator (TFE).

The distillation section advantageously uses a distillation column or several separate items of equipment. Preferably, the distillation section of optional step f) is advantageously operated in a distillation column, at a column head temperature preferably between 25 and 60° C., preferentially between 45 and 55° C., for example about 50° C., preferably at a column bottom temperature of between 80 and 120° C., preferentially between 105 and 115° C., for example about 110° C., preferably at a pressure of between 0.001 and 0.05 MPa, preferentially between 0.005 and 0.02 MPa and more preferably between 0.008 and 0.012 MPa, and preferably with a reflux ratio of between 0.01 and 0.50, more preferably between 0.05 and 0.10.

Thus, the aqueous raffinate 5 produced in step b) and comprising water and DMSO and optionally the water-DMSO mixture recovered in the optional dehydration step are evaporated, then the gas phase is recovered and distilled, preferably under vacuum, so as to produce a DMSO-rich residue 16 on the one hand and a water-rich distillate 15 (corresponding to the aqueous effluent) on the other hand. The term “rich” here means more than 95% by weight, preferably more than 98% by weight. Part or all of the water-rich distillate, or aqueous effluent, may advantageously be recycled into step c) as aqueous solvent to perform the backwashing step and/or into the hydrodistillation step e) as aqueous stream. Said water-rich distillate may also be totally or partly recycled as water introduced into step a).

The DMSO-rich residue may advantageously be introduced into the optional dehydration step, either directly or after distillation, allowing any heavy products that may accumulate to be removed.

The examples and figures appended and described below illustrate the invention without limiting its scope.

FIG. 1 illustrates a particular embodiment of the process according to the invention. The feedstock 1 containing 5-HMF, DMSO and humins is sent to step a) and placed in contact with the intermediate aqueous back-extract 9 from step c), after which the precipitated humins 2 are removed from the mixture by liquid-solid filtration. The aqueous mixture 3 obtained on conclusion of step a) is sent to extraction step b) and placed in contact with an extraction solvent 4 recycled from steps d) and e), so as to extract the 5-HMF from the aqueous mixture using the extraction solvent and obtain an aqueous raffinate 5 and an intermediate organic extract 6. The intermediate organic extract 6 is placed in contact with an aqueous solvent 7 in the backwashing step c). The organic raffinate 8 obtained is concentrated in step d) by removing the stream 11 recycled into step b). The concentrated organic extract 10 obtained on conclusion of step d) is treated in a hydrodistillation step e) so as to remove the residual organic solvent 13, recycled into step b), and to obtain an aqueous solution 12 of 5-HMF.

FIG. 2 illustrates another particular embodiment of the process according to the invention, which differs from that of FIG. 1 in that the process comprises a step f) of treating the water-DMSO mixtures produced within the process, and in particular the aqueous raffinate 5, to produce an aqueous effluent 15, part of which is recycled into step c) as aqueous solvent 7 and part of which is recycled into hydrodistillation step e) as aqueous stream 14, a DMSO-rich residue 16 and an impurities stream 17.

### EXAMPLES

### Example 1: Preparation of an Organic Extract 8 According to the Invention

In order to demonstrate some of the advantages of the process according to the invention, the results of implementing the process according to FIG. 1 are presented here.

An acid catalyst, methanesulfonic acid, is mixed with DMSO, such that the mole ratio with the sugar feedstock (catalyst/sugar feedstock) is 1 mol %, and they are brought to a temperature of 120° C. Fructose is introduced in the form of an aqueous solution, at 70% by weight of sugar (syrup), in a DMSO/fructose mass ratio of 2.3. The pressure is maintained at 0.035 MPa. Under these pressure and temperature conditions, the reaction medium is above the bubble point of the mixture, so the vapor phase can be withdrawn from the reactor, and condensed to form the condensates. The sugar dehydration step is performed batchwise, by addition of feedstock progressively over a 2-hour period. The reaction medium is maintained at the temperature and pressure indicated above for a further 2 h after the end of addition.

The liquid effluent resulting from the dehydration step contains 74% by weight of DMSO, 21% by weight of 5-HMF and 3% by weight of water, giving a molar yield of 5-HMF relative to engaged fructose of 81%. Polymeric compounds (called humins) which are soluble in the reaction medium were formed in an amount of 5% by weight. During this dehydration step, a water-DMSO mixture is recovered in the vapor phase. Said water-DMSO mixture has a composition of 32% by weight of DMSO and 68% water. This water-DMSO mixture is distilled under vacuum to produce water containing only traces of DMSO.

The liquid effluent from the dehydration step corresponding to feedstock 1 is engaged in a step a) of placing in contact with a water-containing stream, at room temperature, so as to obtain a mixture which contains a DMSO/water mass ratio equal to 1.

The mixture from step a) is subjected to a liquid-solid separation step, on a Büchner filter equipped with a polypropylene gauze filter with a pore size of 10 μm. This liquid-solid separation step is performed at room temperature. During the liquid-solid separation step, 7.5 g of a “humins” solid residue/kg of filtered mixture are recovered, along with a homogeneous liquid phase corresponding to aqueous mixture 3. Aqueous mixture 3 is composed of 43% by weight of DMSO, 12% by weight of 5-HMF and 43% by weight of water, and comprises impurities (about 2% by weight of humins).

The aqueous mixture 3 resulting from step a) is subjected to a countercurrent liquid-liquid extraction step b) in a stirred glass column (Kühni or ECR type) comprising eight sections 225 mm high and with an inside diameter of 32 mm, and also a lower decanter and an upper decanter. The useful height is about 1.8 m and the total column height is 2.60 m. The total volume is about 3 liters. The organic extraction solvent is methyl isobutyl ketone (MIBK). Said aqueous mixture 3 is introduced into the upper part of the device and dispersed in the ascending organic phase. The column inlet flow rates are set at 2.2 kg/h for the DMSO-water phase and 4.1 kg/h for the organic extraction solvent. The proportion (weight/weight) of MIBK solvent is 1.9 relative to the aqueous mixture 3 resulting from step a). In this step b), the temperature is 20° C. and the stirring speed is 300 rpm.

On conclusion of step b), a 5-HMF-depleted aqueous raffinate 5 is recovered, containing about 48% by weight of water, 48.5% by weight of DMSO, 0.4% by weight of 5-HMF, 1.8% by weight of MIBK, and humin impurities, and an intermediate organic extract 6 enriched in furan compounds containing 2.8% by weight of DMSO, 5.9% by weight of 5-HMF (a 5-HMF/DMSO weight ratio of about 68/32), and 91.3% by weight of MIBK. The extraction yield is 97% for 5-HMF and 13% for DMSO.

The intermediate organic extract 6 resulting from liquid-liquid extraction step b) is subjected to a backwashing step c) in the same extraction device (Kühni type stirred column or ECR). Said organic extract is dispersed in the pure water phase at 21.5° C. The column inlet flow rates are set at 5 kg/h for the organic extract and 1.5 kg/h for the aqueous phase. The proportion (weight/weight) of water introduced as aqueous backwashing solvent relative to the intermediate organic extract is 0.3.

On conclusion of backwashing step c), a DMSO-enriched intermediate aqueous back-extract 9 is recovered, containing 86% by weight of water, 7% by weight of DMSO, 5% by weight of 5-HMF and 2% by weight of MIBK, and an organic raffinate 8, containing 4.3% by weight of 5-HMF, 0.092% by weight of DMSO (i.e. 2.1% by weight of DMSO relative to the weight of 5-HMF) and 88% by weight of MIBK, giving a backwashing yield of 27% by weight for 5-HMF and 95% by weight for DMSO.

### Example 2: Implementation of Steps d) and e) According to the Invention

The organic raffinate 8 produced according to Example 1 is sent to the concentration step d). The solvent is evaporated off under vacuum. The temperature of the liquid is set at 60° C., and the vacuum pressure is set at 0.02 MPa.

Step d) is performed with a vaporization mass ratio of 95%, corresponding to the mass of vaporized organic solvent relative to the engaged mass of organic raffinate from step c). The concentrated organic extract obtained on conclusion of step d) has a mass content of 84% by weight of 5-HMF, 2% by weight of DMSO and 9% by weight of MIBK. The 5-HMF content of the concentrated organic extract (84% by weight) is in accordance with the expected value (at least 40% by weight and not more than 95% by weight), as is its residual solvent content of 11% by weight (sum of 9% MIBK +2% DMSO), which is in accordance with the expected value (at least 5% by weight and not more than 60% by weight). The concentrated organic extract obtained on conclusion of step d) also comprises humin impurities (5% by weight). The recovered distillate essentially contains MIBK and water, eliminated in the form of an azeotrope with the MIBK, which separates into two immiscible phases on condensation.

The concentrated organic extract resulting from step d) is placed in contact with pure water, with a water/concentrated extract mass proportion of 0.95, and then sent to a hydrodistillation step e) performed by distillation. Hydrodistillation step e) is performed at a column bottom temperature of 35° C., and under a vacuum of 0.01 MPa, so as to facilitate removal of residual MIBK organic solvent, in the form of a water/MIBK azeotrope without degradation of 5-HMF. The aqueous 5-HMF solution obtained on conclusion of step e) has a composition of 45% by weight of 5-HMF, 53.3% by weight of water, 1% by weight of DMSO (i.e. 2.2% by weight of DMSO relative to the weight of 5-HMF) and 0.7% by weight of MIBK.

### Example 3: Implementation of Steps d) and e) According to the Invention

The organic raffinate 8 produced according to Example 1 is sent to the concentration step d). The solvent is evaporated off under vacuum. The temperature of the liquid is set at 60° C., and the vacuum pressure is set at 0.02 MPa.

Step d) is performed with a vaporization mass ratio of 93%, corresponding to the mass of vaporized organic solvent relative to the engaged mass of organic raffinate from step c). The concentrated organic extract obtained on conclusion of step d) has a mass content of 59% by weight of 5-HMF, 1% by weight of DMSO and 34% by weight of MIBK. The 5-HMF content of the concentrated organic extract (59% by weight) is in accordance with the expected value (at least 40% by weight and not more than 95% by weight), as is its residual solvent content of 35% by weight (sum of 34% MIBK+1% DMSO), which is in accordance with the expected value (at least 5% by weight and not more than 60% by weight). The concentrated organic extract obtained on conclusion of step d) also comprises humin impurities (about 6% by weight). The recovered distillate essentially contains MIBK and water, eliminated in the form of an azeotrope with the MIBK, which separates into two immiscible phases on condensation.

The concentrated organic extract resulting from step d) is placed in contact with pure water, with a water/concentrated extract mass proportion of 0.83, to proceed to a hydrodistillation step e) performed by distillation. Hydrodistillation step e) is performed at a column bottom temperature of 49° C., and under a vacuum of 0.008 MPa, so as to facilitate removal of residual MIBK organic solvent, in the form of a water/MIBK azeotrope without degradation of 5-HMF. The aqueous 5-HMF solution obtained on conclusion of step e) has a composition of 42% by weight of 5-HMF, 56.5% by weight of water, 0.9% by weight of DMSO (i.e. 2.1% by weight of DMSO relative to the weight of 5-HMF) and 0.6% by weight of MIBK.

### Example 4: Implementation of Steps d) and e)

The organic raffinate 8 produced according to Example 1 is sent to the concentration step d). The solvent is evaporated off under vacuum. The temperature of the liquid is set at 60° C., and the vacuum pressure is set at 0.02 MPa.

Step d) is performed with a vaporization mass ratio of 70%, corresponding to the mass of vaporized organic solvent relative to the engaged mass of organic raffinate from step c). The concentrated organic extract obtained on conclusion of step d) has a mass content of 15% by weight of 5-HMF, 0.5% by weight of DMSO and 79% by weight of MIBK. The concentrated organic extract obtained on conclusion of step d) also comprises humin impurities (5.5% by weight). The concentrated organic extract obtained on conclusion of step d) still comprises 79.5% by weight of residual solvent (79% of MIBK+0.5% of DMSO). The recovered distillate essentially contains MIBK and water, eliminated in the form of an azeotrope with the MIBK, which separates into two immiscible phases on condensation.

The 5-HMF content of the concentrated organic extract of 15% by weight is lower than expected (at least 40% by weight and not more than 95% by weight). The residual solvent content of the concentrated organic extract is 79.5% by weight (sum of 79% MIBK+0.5% DMSO) and thus well above the expected value (at least 5% by weight and not more than 60% by weight).

The concentrated organic extract resulting from step d) is placed in contact with pure water, with a water/concentrated extract mass proportion of 0.45, to proceed to a hydrodistillation step e) performed by distillation. Hydrodistillation step e) is performed at a column bottom temperature of 49° C., and under a vacuum of 0.008 MPa, so as to facilitate removal of residual MIBK organic solvent, in the form of a water/MIBK azeotrope without degradation of 5-HMF.

However, the placing in contact of the concentrated organic extract obtained on conclusion of the concentration step d), which still comprises 79.5% by weight of organic solvent (a value well above the targeted 60% by weight limit), induces phase separation of the liquid phase, to generate an aqueous phase and an organic phase which are mutually immiscible, not allowing hydrodistillation step e) to be performed.

1. A process for producing an aqueous solution of 5-hydroxymethylfurfural (5-HMF), said process comprising the following steps:
a step a) of placing a feedstock (1) comprising 5-HMF and dimethyl sulfoxide (DMSO) in contact with at least a fraction of an intermediate aqueous back-extract (9), advantageously obtained from step c), so as to obtain at least one aqueous mixture (3),
a step b) of liquid-liquid extraction of the mixture (3) obtained on conclusion of step a) in the presence of an extraction solvent so as to produce an aqueous raffinate (5) and an intermediate organic extract (6), and then
a step c) of backwashing with an aqueous solvent (7), so as to produce the intermediate aqueous back-extract (9) and an organic raffinate (8) comprising 5-HMF and an organic solvent,
a step d) of concentrating the organic raffinate (8) obtained from step c) by removing at least part of the organic solvent, producing a concentrated organic extract (10), comprising 5-HMF, preferably in a content greater than or equal to 40% by weight, and residual organic solvent, preferably in a content less than or equal to 60% by weight, and a stream (11) comprising organic solvent,
a hydrodistillation step e) performed by distilling the concentrated organic extract (10) obtained from step d) in the presence of water, to produce an aqueous solution (12) of 5-HMF and a stream (13) comprising organic solvent.

2. The process as claimed in claim 1, in which the intermediate organic extract (6) from step b) feeds the backwashing step c).

3. The process as claimed in claim 1, in which the vaporization of the organic solvent in step d) is performed at atmospheric pressure or under vacuum, preferably at a pressure of between 0.1 and 0.01 MPa, preferentially under vacuum at a pressure of between 0.09 and 0.01 MPa.

4. The process as claimed in claim 1, in which the temperature of the liquid in step d) is kept below 130° C., preferably kept below 100° C., more preferably kept below 70° C.

5. The process as claimed in claim 1, in which the concentrated organic extract (10) obtained on conclusion of step d) has a 5-HMF content of at least 40% by weight, preferably at least 50% by weight, more preferably at least 60% by weight, relative to the weight of concentrated organic extract, and preferably not more than 95% by weight, preferably not more than 90% by weight, and more preferably not more than 85% by weight, relative to the weight of concentrated organic extract, and a residual organic solvent content of at least 5% by weight, preferably at least 10% by weight, relative to the weight of concentrated organic extract, and preferably not more than 60% by weight, preferably not more than 50% by weight, more preferably not more than 40% by weight, relative to the weight of concentrated organic extract.

6. The process as claimed in claim 1, in which an aqueous stream (14) feeds the hydrodistillation step e).

7. The process as claimed in claim 1, in which step e) is performed at atmospheric pressure or under vacuum and in particular at a pressure of between 0.1 MPa and 0.001 MPa, preferably under vacuum at a pressure of between 0.08 and 0.005 MPa.

8. The process as claimed in claim 1, in which step e) is performed in a distillation column, preferably at a column bottom temperature of less than 140° C., preferably less than 130° C., preferably less than 120° C., preferably less than 110° C. and preferably less than 100° C.

9. The process as claimed in claim 1, in which the extraction solvent (4) is chosen from dichloromethane, diethyl ether, diisopropyl ether, methyl ethyl ketone, methyl isopropyl ketone, methyl isobutyl ketone, thiophene, anisole and toluene, very preferably methyl isobutyl ketone.

10. The process as claimed in claim 1, in which the weight ratio (weight/weight) of aqueous solvent (7) relative to the intermediate organic extract (6) in the backwashing step c) is from 0.04 to 5, preferably between 0.07 and 3, more preferably between 0.1 and 1.

11. The process as claimed in claim 1, comprising a step of dehydrating the sugars to 5-HMF, upstream of step a), preferably by placing a sugar feedstock comprising one or more sugars in contact with DMSO and an acidic dehydration catalyst, preferably at a temperature of between 50 and 150° C., preferably between 60 and 140° C., preferably between 70 and 130° C. and more preferably between 80 and 120° C., and preferably at a pressure of between 1 and 0.001 MPa, more preferably between 0.1 and 0.01 MPa.

12. The process as claimed in claim 1, comprising a step f) of treating the water-DMSO mixtures produced within the process, allowing the production of an aqueous effluent, which may be used totally or partly in the backwashing step c), and/or in step e).
